(19) United States
(12) Patent Application Publication
(10) Pub. No.: US 2015/0241426 A1
(43) Pub. Date: Aug. 27, 2015

(54) METHOD FOR DETECTING INFECTION BY DETECTING INFECTIOUS AGENT ANTIGENS

(71) Applicant: RareCyte, Inc., Seattle, WA (US)

(72) Inventors: Eric Kaldjian, Ann Arbor, MI (US); Joshua Nordberg, Bainbridge Island, WA (US)

(73) Assignee: RareCyte, Inc., Seattle, WA (US)

(21) Appl. No.: 14/618,103

(22) Filed: Feb. 10, 2015

Related U.S. Application Data
(60) Provisional application No. 61/945,029, filed on Feb. 26, 2014.

(51) Int. Cl. G01N 33/569

(52) U.S. Cl. CPC G01N 33/56911; G01N 2469/10

(57) ABSTRACT

This disclosure is directed to a method for detecting an infection within a suspension without having to directly detect the infectious agent or without having to wait until symptoms of infection are present within the host. Once a sample is obtained, infectious agent-specific antibodies conjugated with labeling molecules are added to the biological sample. The infection may be detectable despite the absence of the infectious agent during detection because antigens released by the infectious agent may still be present on or within a biological component of the sample. The sample may undergo imaging to detect the infection.

### CROSS-REFERENCE TO A RELATED APPLICATION

This application claims the benefit of Provisional Application No. 61/945,029, filed Feb. 26, 2014.

### TECHNICAL FIELD

This disclosure relates generally to a method for analyzing a suspension and, in particular, to analyzing a suspension for an infection without detecting the infectious agent.

### BACKGROUND

Suspensions often include materials of interest that are difficult to detect, extract and isolate for analysis. For instance, whole blood is a suspension of materials in a fluid. The materials include billions of red and white blood cells and platelets in a proteinaceous fluid called plasma. Whole blood is routinely examined for the presence of abnormal organisms, such as parasites, bacteria, fungi, and viruses, including HIV, cytomegalovirus, hepatitis C virus, and Epstein-Barr virus, or cells such as atypical inflammatory cells or malignant cells. However, materials of interest composed of particles that occur in very low numbers are especially difficult if not impossible to detect and analyze using many existing techniques. Consider, for instance, Borrelia burgdorferi, the causative agent of Lyme disease, circulating in the bloodstream. The ability to accurately detect and analyze Borrelia burgdorferi is of particular interest to clinicians, but Borrelia burgdorferi are difficult to detect.

As a result, practitioners, researchers, and those working with suspensions continue to seek systems and methods to more efficiently and accurately detect infection without directly detecting the infectious agent or waiting until symptoms of infection are present within the host.

### DESCRIPTION OF THE DRAWINGS

FIG. 1 shows a flow diagram of an example method for testing for infection.

FIG. 2 shows an example white blood cell having been labeled with two different fluorescent labeling molecules conjugated to Borrelia burgdorferi antibodies and a nucleic acid marker; and an example Borrelia burgdorferi having been labeled with two different fluorescent labeling molecules conjugated to Borrelia burgdorferi antibodies.

FIG. 3A shows monoclonal anti-Borrelia burgdorferi antibody labeling from FIG. 2.

FIG. 3B shows polyclonal anti-Borrelia burgdorferi antibody labeling from FIG. 2.

FIG. 3C shows nuclear labeling of the white blood cell from FIG. 2.

FIGS. 4A-4C show positive labeling of Borrelia burgdorferi antigens with monoclonal and polyclonal antibodies.

### DETAILED DESCRIPTION

This disclosure is directed to a method for detecting an infection within a suspension without having to directly detect the infectious agent or without having to wait until symptoms of infection are present within the host. Once a sample is obtained, infectious agent-specific antibodies bound with labeling molecules are added to the biological sample. The infection may be detectable without directly detecting the infectious agent because antigens released by the infectious agent may still be present on or within a biological component of the sample. The sample may undergo imaging to detect the infection.

In the following description, the term “biological sample” is used to describe a specimen to be analyzed, such that the specimen may be a suspension, a portion of the suspension, or a component of the suspension. For example, when the suspension is anticoagulated whole blood, the biological sample may be the anticoagulated whole blood (i.e. a suspension), the buffy coat (i.e. a portion of the suspension), or a cell, cell fragment, bacterium, parasite, or virus (i.e. a component of the suspension).

In the following description, the term “infection” is used to describe an association of a host with an infectious agent. The infection may or may not result in an infectious disease, an immune response, and/or physical symptoms within the host.

In the following description, the term “infectious agent” is used to describe an organism which associates with a host, which may lead to an infectious disease within the host. The infectious agent may include, but is not limited to, viruses, viroids, prions, microorganisms (e.g. bacteria), nematodes (e.g. roundworms and pinworms), fungi (e.g. ringworm), and other macroparasites (e.g. tapeworms).

In the following description, the term “antigen” is used to describe a substance that is foreign to the host and that may be native to the infectious agent. An antigen may include, but is not limited to, a protein, a saccharide (e.g. mono-, di-, or polysaccharide), a lipid, a nucleic acid, a remnant of product of the infectious agent, or the like.

### Method

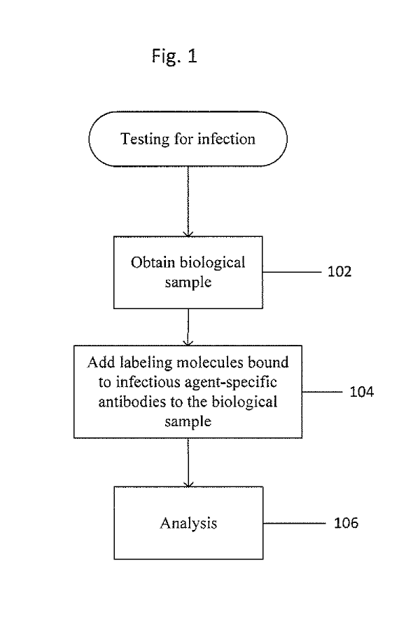

FIG. 1 shows a flow diagram of an example method for detecting an infection caused by an infectious agent without directly detecting the infectious agent. In block 102, a biological sample suspected of including an infection, such as anticoagulated whole blood, is obtained, such as by venipuncture. Alternatively, a biological suspension may be obtained and then processed so as to extract the desired biological sample from the non-desired components of the biological suspension (i.e. huffy coat may be isolated from other blood components by undergoing density-based separation; or, a single cell may be obtained from the buffy coat by being picked by a cell picker or by being separated by flow cytometry). In block 104, labeling then occurs, whereby many labeling solutions, each including a different labeling molecule conjugated to infectious agent-specific antibodies, may be added to the biological sample. A solution including a nucleic acid label may also be added to the sample. The infectious agent-specific antibodies attach to complementary antigens that were released by the infectious agent. The biological component may also be labeled with many labeling solutions, each including a different labeling molecule conjugated to biological component-specific antibodies. In block 106, the biological sample then undergoes analysis to determine the presence or absence of the infection, which may be done by imaging, such as with a microscope, a scanner, or the like, by flow cytometry, or by other immunodetection methods, including, but not limited to, an ELISA assay. Analysis, such as imaging, may be done in fluorescence, bright field, or dark field.

When the infectious agent releases the antigens, the antigens may be taken up by a biological component (such as a cell) of the biological sample, the antigens may bind to a biological component of the biological sample, or the antigens may be dispersed throughout or within a biological component that the infectious agent has invaded (and may have subsequently exited). The infection may be detectable without directly detecting the infectious agent because the antigens may be present on or within the biological component. The infectious agent may be present on or within the biological component but not visible to detection; the infectious agent may be absent from the biological component; or the infectious agent may be present on or within the biological component and visible to detection, though the infectious agent may be distinctly or indistinctly visible. The infectious agent-specific antibodies bound to labeling molecules may still remain attached to complementary antigens that were released by the infectious agent, thereby rendering the infection detectable when imaged.

The labeling step may occur within a tube, with a float and tube system, on a slide, or within or on any appropriate vessel, such as a well plate, for storing the biological sample. The labeling step may be performed before or after fixation and/or before or after permeabilization. The biological sample may be imaged while in the vessel or may be transferred to a different vessel that is appropriate for imaging.

Examples of suitable labeling molecules include, but are not limited to, fluorescent molecules including, but not limited to, quantum dots; commercially available dyes, such as fluorescein, Hoechst, FITC (“fluorescein isothiocyanate”), R-phycoerythrin (“PE”), Texas Red, allophycocyanin, Cy5, Cy7, cascade blue, DAPI (“4′,6-diamidino-2-phenylindole”) and TRITC (“tetramethylrhodamine isothiocyanate”); combinations of dyes, such as CY5PE, CY7APC, and CY7PE; reaction-confirmation probes; metal-conjugated antibodies; immunohistochemical stains (e.g. chromogenic stains); and synthesized molecules, such as self-assembling nucleic acid structures (e.g. DNA barcodes).

### Biological Applications

For the sake of convenience, the infectious agent discussed herein is Borrelia burgdorferi, which is the causative agent of Lyme disease, though the infectious agent can be viruses, viroids, prions, other microorganisms, nematodes (e.g. roundworms and pinworms), fungi (e.g. ringworm), and other macroparasites (e.g. tapeworms). For the sake of convenience, the biological suspension discussed herein is blood, though the biological suspension can be urine, bone marrow, cystic fluid, ascites fluid, stool, semen, cerebrospinal fluid, an aspirate (such as a fine needle aspirate or nipple aspirate fluid) saliva, amniotic fluid, vaginal secretions, mucus membrane secretions, aqueous humor, vitreous humor, vomit, and any other physiological fluid or semi-solid. Furthermore, for the sake of convenience, the biological sample discussed herein is buffy coat, though the biological sample may be one of the suspensions listed above; the biological sample may be another portion of the suspension, such as plasma; or, the biological sample may be a component of the sample, such as, a cell.

First, anticoagulated whole blood is obtained. The whole blood may be added to a tube or the whole blood may be collected directly into the tube. A polyclonal antibody to whole cell Borrelia burgdorferi lysate conjugated to FITC, a monoclonal antibody to OspB protein (i.e. an approximately 30 kilo Dalton Borrelia burgdorferi protein) conjugated to Alexa555, and a Hoechst dye may all be added to the tube for labeling purposes. The polyclonal antibody may also be a polyclonal antibody to whole Borrelia burgdorferi organism. A float may also be added to the tube already containing the whole blood. The tube, the float, and the whole blood undergo density-based separation, such as by centrifugation, thereby permitting separation of the whole blood into density-based fractions along an axial position in the tube based on density. The blood separates into three fractions along an axial position in the tube based on density, with red blood cells located on the bottom, plasma located on top, and buffy coat located in between. The density of the float can be selected so that the float settles at the same axial position of the buffy coat. The buffy coat can be trapped within an area between the float and the primary vessel.

A seal may then be created between the float and the tube to prevent fluids and other components, such as cells, from moving past the seal in any direction within the tube. The seal may also inhibit float movement within the tube after the initial centrifugation step has been completed. To create the seal, a sealing ring is clamped around the tube at the desired location along the axial length of the tube and pressure is applied inwardly to collapse the tube towards the float until the seal is formed.

After forming the seal, the plasma is removed from the tube, such as by pipetting, suctioning, pouring, or the like. A collector and processing vessel system is then added to the tube. The collector includes a primary body including a first end and an opposing second end, the primary body including a concave opening in the second end that narrows to an apex within the primary body, and a cavity with an opening at the first end; and a cannula that extends from the apex into the cavity, the cannula to provide an opening between the concave opening and the cavity. The processing vessel may be an Eppendorf tube. The processing vessel also includes a displacement fluid that has a density greater than the buffy coat. The system is then re-centrifuged. The displacement fluid flows from the processing vessel into the tube and, having a density greater than the buffy coat, displaces the buffy coat upwards within the tube, through the collector, and into the processing vessel during the re-centrifugation step.

Alternatively, a clearing fluid may be added to the tube before the collector. The clearing fluid is the densest of the displacement fluids and displaces the fractions located above the sealing ring.

The processing vessel, now including the buffy coat, may then be removed from the collector. The buffy coat may then be diluted with a buffer, such as phosphate buffered saline, tris buffered saline, tris with hydrochloride, Ringer's solution, or the like. The dilute buffy coat may then be added to a microscope slide. The buffy coat may be attached (such as with an attachment solution) or fixed (such as with a fixative) to the microscope slide and may then undergo subsequent imaging on a fluorescent microscope. Any component, such as a cell or Borrelia burgdorferi, may be extracted from the slide, such as by picking with a cell picker, and undergo further processing, such as an amplification reaction (e.g. polymerase chain reaction).

To image the buffy coat, the microscope slide (or tube) is illuminated with one or more wavelengths of excitation light from a light source, such as red, blue, green, and ultraviolet. The excitation light is focused by an objective onto the buffy coat on the microscope slide. The different wavelengths excite different labeling molecules, causing the labeling molecules to emit light at lower energy wavelengths. A portion of the light emitted by the labeling molecules is captured by the objective and transmitted to a detector that generates images that are processed and analyzed by a computer or associated software or programs. The images formed from the emission light of each labeling molecule can be overlaid when a plurality of labeling molecules are excited and emit light. The images may then be analyzed to detect and characterize an infection within the buffy coat based on the antigens present within the sample.

Alternatively, imaging may be done directly in the tube after completion of the initial centrifugation step.

The images may also provide for location determination for picking, so as to select and isolate a specific analyte for further processing, such as extracellular and intracellular analysis including intracellular protein labeling; nucleic acid analysis, including, but not limited to, DNA arrays, expression arrays, protein arrays, and DNA hybridization arrays; in situ hybridization (“ISH”—a tool for analyzing DNA and/or RNA, such as gene copy number changes); polymer chain reaction (“PCR”); reverse transcription PCR; or branched DNA (“bDNA”—a tool for analyzing DNA and/or RNA, such as mRNA expression levels) analysis.

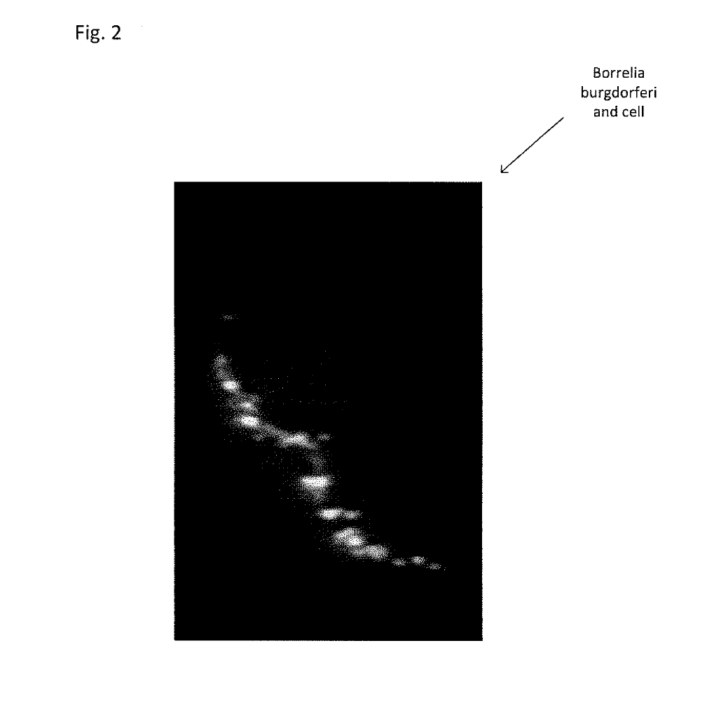

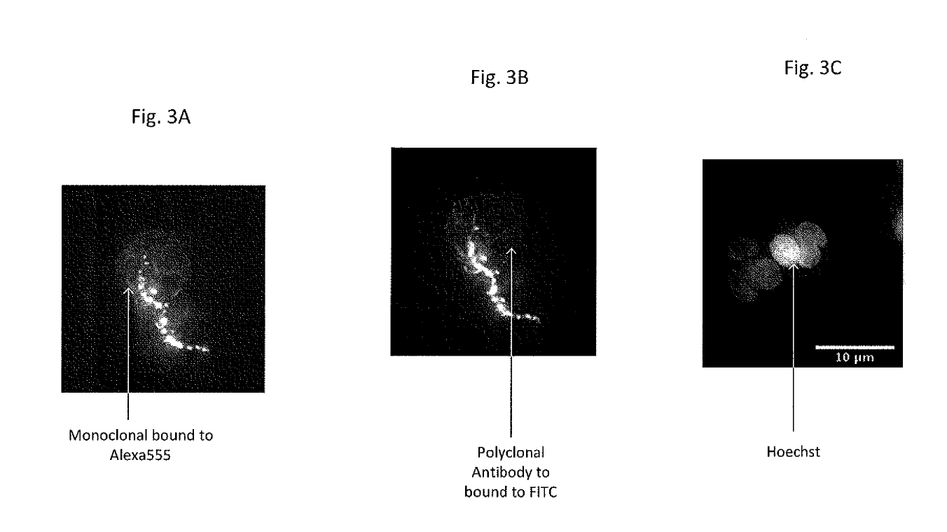

FIG. 2 shows a cell with an associated Borrelia burgdorferi labeled with three different fluorescent labeling molecules. A polyclonal Borrelia burgdorferi antibody conjugated to FITC (as shown in FIG. 3B), a monoclonal Borrelia burgdorferi antibody conjugated to Alexa555 (as shown in FIG. 3A), and a Hoechst dye (as shown in FIG. 3C) were all added to the tube after collecting a blood sample. It is important to note that the cell associated with the Borrelia burgdorferi is fluorescently labeled even though the polyclonal and monoclonal antibodies are specific to Borrelia burgdorferi. The Borrelia burgdorferi may have released antigens into or onto the cell. Though the Borrelia burgdorferi may be partially within the cell, when the Borrelia burgdorferi is expelled from or removes itself from the cell and the antigens are still present within the cell, the cell may still appear under fluorescent imaging due to the fluorescent molecules bound to the Borrelia burgdorferi-specific antibodies which bind to the antigens. So even though the Borrelia burgdorferi is no longer associated with the cell during detection, the infection may be detected due to the antigens that were left within or on the cell by the Borrelia burgdorferi.

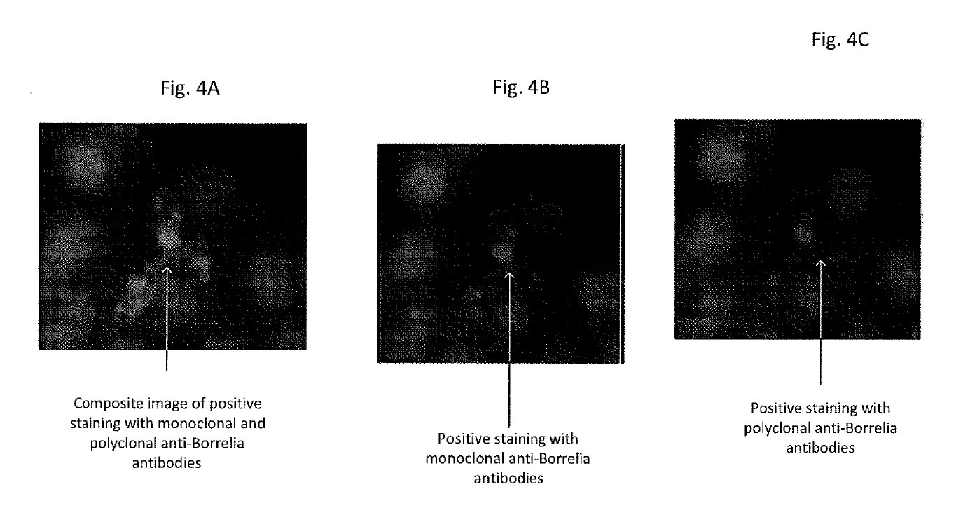

FIGS. 4A-4C show positive labeling of Borrelia burgdorferi antigens with monoclonal and polyclonal antibodies. FIG. 4A shows a composite image of positive staining with monoclonal and polyclonal anti-Borrelia burgdorferi antibodies. FIG. 4B shows an image with positive staining with monoclonal anti-Borrelia burgdorferi antibodies. FIG. 4C shows an image with positive staining with polyclonal anti-Borrelia burgdorferi antibodies. It is important to note that the objects labeled are not the Borrelia burgdorferi but are rather antigens associated with the Borrelia burgdorferi. So even though the Borrelia burgdorferi is no longer associated with the sample during detection, the infection may be detected due to the antigens that were left within the sample by the Borrelia burgdorferi.

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific embodiments are presented by way of examples for purposes of illustration and description. They are not intended to be exhaustive of or to limit this disclosure to the precise forms described. Many modifications and variations are possible in view of the above teachings. The embodiments are shown and described in order to best explain the principles of this disclosure and practical applications, to thereby enable others skilled in the art to best utilize this disclosure and various embodiments with various modifications as are suited to the particular use contemplated. It is intended that the scope of this disclosure be defined by the following claims and their equivalents:

I/We claim:

1. A method for detecting an infection of a host by an infectious agent, the method comprising:
adding a first labeling solution including a first labeling molecule conjugated to a first infectious agent-specific antibody to a sample suspected of including the infection to bind to an infectious agent antigen within the sample; and,
analyzing the sample to verify the infection based on the presence of the infectious agent antigen.

2. The method of claim 1, wherein the first infectious agent-specific antibody is a polyclonal antibody to whole cell Borrelia burgdorferi lysate or whole Borrelia burgdorferi organism.

3. The method of claim 2, further comprising adding a second labeling solution including a second labeling molecule conjugated to a second infectious agent-specific antibody to the sample to bind to the infectious agent antigen.

4. The method of claim 3, wherein the second infectious agent-specific antibody is a monoclonal Borrelia burgdorferi antibody.

5. The method of claim 4, the monoclonal Borrelia burgdorferi antibody to bind to OspB protein.

6. The method of claim 1, further comprising adding a second labeling solution including a second labeling molecule conjugated to a second infectious agent-specific antibody to the sample to bind to the infectious agent antigen.

7. The method of claim 6, wherein the second infectious agent-specific antibody is a monoclonal Borrelia burgdorferi antibody.

8. The method of claim 7, the monoclonal Borrelia burgdorferi antibody to bind to OspB protein.

9. The method of claim 1, further comprising adding a solution including a nucleic acid marker to the sample.

10. The method of claim 1, wherein the infectious agent antigen is on or within a biological component of the sample, and wherein the biological component is not the infectious agent.

11. The method of claim 1, wherein the infectious agent is Borrelia burgdorferi.

12. The method of claim 11, wherein the infectious agent antigen is on or within a biological component of the sample, and wherein the biological component is a eukaryotic cell or a fragment of a cell.

13. The method of claim 12, further comprising adding at least one biological component labeling solution to label the biological component, each of the at least one biological component labeling solutions including a labeling molecule conjugated to a biological component-specific antibody,
wherein no two biological component labeling solutions have the same labeling molecule or biological component-specific antibody, and
wherein no biological labeling molecule or infectious agent labeling molecule are the same.

14. The method of claim 1, wherein the sample is obtained by extracting the sample from a biological suspension.

15. The method of claim 14, wherein the sample is buffy coat and the biological suspension is anticoagulated whole blood.

16. The method of claim 1, wherein the infectious agent is not present or not detectable on or within the biological component during the analysis step.

17. The method of claim 1, wherein the infectious agent is detectable or present during the analysis step.

18. The method of claim 17, wherein analyzing the sample confirms the presence of the infectious agent when the infectious agent is neither directly detectable nor indistinctly detectable.

19. The method of claim 1, further comprising adding at least two more labeling solutions to the sample, wherein no two labeling solutions have the same labeling molecule or infectious disease-specific antibody.

20. The method of claim 1, wherein the analyzing step may be performed by imaging, an ELISA assay, or flow cytometry.
